(19) United States
(12) Patent Application Publication
(10) Pub. No.: US 2012/0048964 A1
(43) Pub. Date: Mar. 1, 2012

(54) SCENT-RELEASING APPARATUS AND METHOD OF MAKING SAME

(76) Inventor: Bryan Bruce Willert, Saint Louis, MO (US)

(21) Appl. No.: 12/862,562

(22) Filed: Aug. 24, 2010

(51) Int. Cl. A61L 9/04

(52) U.S. Cl. 239/57

(57) ABSTRACT

A scent-releasing apparatus is provided. The scent-releasing apparatus includes a housing having at least a front wall, a back wall, and a side wall connecting the front wall to the back wall. The housing defines an inner chamber, wherein at least one of the front wall and the back wall includes a plurality of apertures. At least one cover member is removably attached to at least one of the front wall and the back wall for covering the plurality of apertures, and the housing and the cover member are configured to substantially seal the inner chamber from ambient air. A scent-releasing material is positioned within the inner chamber, and the scent-releasing material is configured to release a scent through the apertures when the at least one cover member is removed from at least one of the front wall and the back wall.

### BACKGROUND OF THE INVENTION

The subject matter described herein relates generally to scent-releasing apparatuses and, more particularly, to a scent-releasing apparatus for repelling insects and a method of making the same.

Many known moth-repelling devices are configured for placement in an enclosure that stores clothing (e.g., a chest, a drawer, or a closet) to prevent moths from entering the enclosure and eating the clothing. At least one of these known moth-repelling devices is deployed with a scent-releasing agent disposed within a container, and the container is configured to permit the scent to be dispersed into the ambient air surrounding the container.

Often, when the device is sold to the consumer, the scent-releasing agent is wrapped in a barrier-film that prevents the scent from being released before the device is to be deployed. As such, the user is required to unwrap (i.e., handle) the scent-releasing agent from the barrier film and place the unwrapped agent into the container, thereby initiating release of the scent from the container and into the ambient air surrounding the container. However, such contact with the scent-releasing agent can be undesirable. Additionally, at least some known devices develop pressure within the container as a result of the vapor that is released by the agent within the container. Yet, many known scent-releasing devices have containers that are not configured to withstand the pressure and have been known to significantly deform as a result, which detracts from the effectiveness and longevity of the scent-releasing device.

As such, it would be useful to provide a scent-releasing device that can be more easily deployed without the user having to handle the scent-releasing agent. Also, it would be useful to provide a scent-releasing device that is configured to better withstand pressure created by the scent-releasing agent.

### BRIEF DESCRIPTION OF THE INVENTION

In one aspect, a scent-releasing apparatus is provided. The scent-releasing apparatus includes a housing having at least a front wall, a back wall, and a side wall connecting the front wall to the back wall. The housing defines an inner chamber, wherein at least one of the front wall and the back wall includes a plurality of apertures. At least one cover member is removably attached to at least one of the front wall and the back wall for covering the plurality of apertures, and the housing and the cover member are configured to substantially seal the inner chamber from ambient air. A scent-releasing material is positioned within the inner chamber, and the scent-releasing material is configured to release a scent through the apertures when the at least one cover member is removed from at least one of the front wall and the back wall.

In another aspect, a scent-releasing apparatus is provided. The scent-releasing apparatus includes a scent-releasing material and a housing having a front wall, a back wall, and a side wall connecting the front wall to the back wall to define an inner chamber. The scent-releasing material is positioned within the inner chamber and is substantially sealed from ambient air. At least one of the front wall and the back wall is curvilinearly contoured with an inner reinforcing wall extending between the front wall and the back wall within the inner chamber.

In another aspect, a scent-releasing apparatus is provided. The scent-releasing apparatus includes a body having a plurality of apertures and a bottom cap welded to the body to define an inner chamber between the body and the bottom cap. A scent-releasing material is disposed within the inner chamber, and at least one substantially fluid impervious cover member is removably attached to the body over the plurality of apertures to substantially seal the inner chamber from ambient airflow into the inner chamber through the plurality of apertures.

### BRIEF DESCRIPTION OF THE DRAWINGS

FIG. 1 is a perspective view of a scent-releasing apparatus;

FIG. 2 is an exploded view of the scent-releasing apparatus shown in FIG. 1;

FIG. 3 is a front view of a housing of the scent-releasing apparatus shown in FIG. 1 with a cover removed;

FIG. 4 is a side view of the housing shown in FIG. 3;

FIG. 5 is a cross-sectional view of the housing shown in FIG. 4 and taken along line 5-5; and

FIG. 6 is a cross-sectional view of the housing shown in FIG. 4 taken along line 6-6.

### DETAILED DESCRIPTION OF THE INVENTION

The exemplary embodiments described herein provide a scent-releasing apparatus that is useful for repelling insects and a method for making the same. More specifically, the exemplary scent-releasing apparatus is useful for repelling moths. The scent-releasing apparatus includes a housing with a bottom cap ultrasonically welded to the housing, and the housing contains a paradichlorobenzene (PDCB) material that sublimes in ambient air at room temperature to release a scent that repels and/or kills moths. A label is removably attached to the housing over a plurality of apertures in the housing for providing a substantially air-tight seal. The label helps prevent ambient air from entering the housing until the label is removed, thereby preventing premature sublimation of the PDCB. Once the label has been removed, ambient air is permitted to flow through the apertures to contact the PDCB, which initiates sublimation of the PDCB, and a moth-repelling scent is then released from the container through the apertures. With the moth-repelling scent being released from the apertures, the scent-releasing apparatus can be hung near clothing within a closet or other suitable space to prevent moths from eating the clothing. Since the apparatus is a reinforced container having a label for creating an air-tight chamber, the PDCB blocks can be placed within the chamber without being wrapped inside a film. Accordingly, a user is not required to handle the PDCB blocks when activating the container. In addition, as explained below, the container is reinforced in certain areas to prevent any pressure related deformation of the container. As used herein, the term “air-tight” refers to preventing a substantial amount of ambient airflow into the housing, thereby inhibiting the ambient air from contacting the scent-releasing material (e.g., the PDCB).

Furthermore, the label is a multi-layered structure that is flexible and is substantially fluid impervious to prevent fluid flow through the label, and the housing has a polished contact surface for improved adherence of the label to the housing. Additionally, a raised rib is provided on the housing around at least a portion of the perimeter of the label to protect the label from damage during storage and/or shipping. Moreover, vapor from the PDCB can pressurize the housing. Thus, the housing has a curvilinear cross-sectional profile and an inner reinforcing wall that prevent the housing from substantially deforming as a result of the pressure. The label is configured to be flexible to permit slight deformations of the housing at high temperatures without detaching from the housing or affecting the air-tight seal of the label. These above-mentioned embodiments inhibit premature sublimation of the PDCB, which increases the shelf life of the scent-releasing apparatus and maintains the production weight of the scent-releasing apparatus.

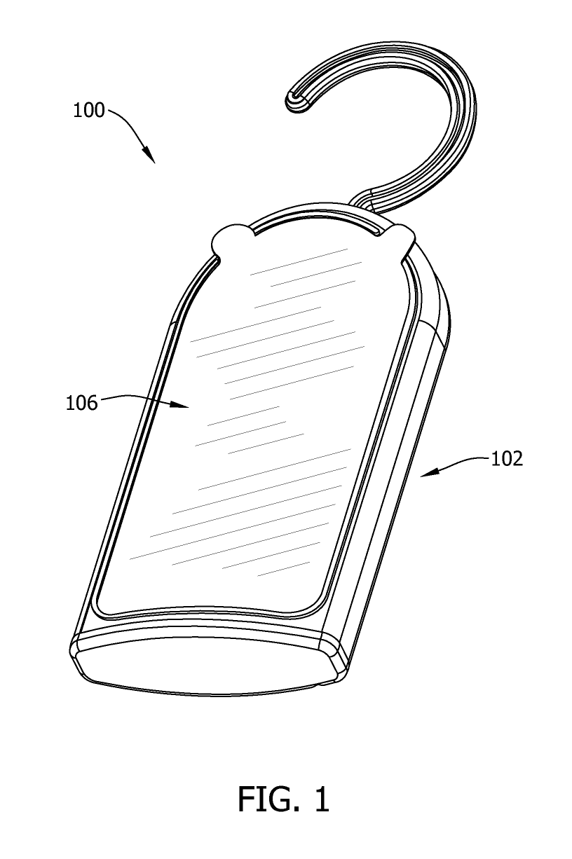

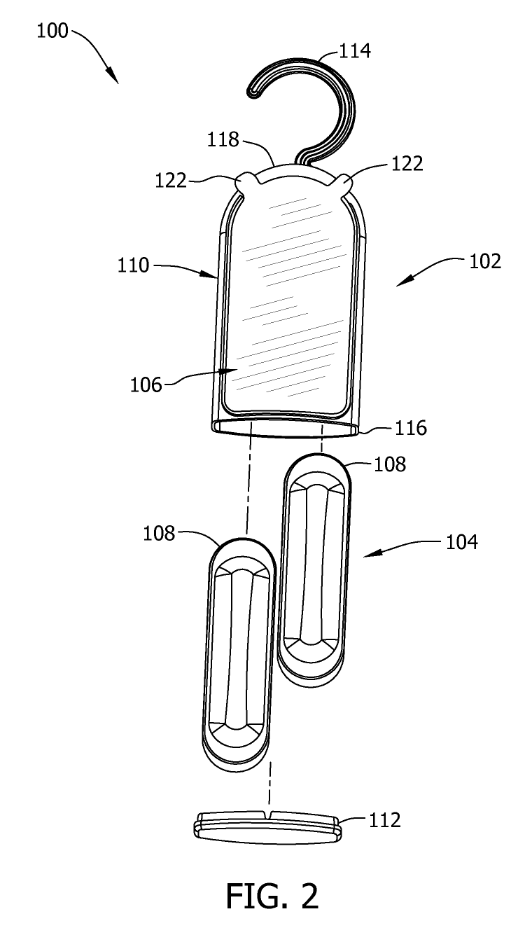

FIG. 1 is a perspective view of an exemplary scent-releasing apparatus 100, and FIG. 2 is an exploded view of scent-releasing apparatus 100. In the exemplary embodiment, scent-releasing apparatus 100 includes a housing 102, a scent-releasing material 104 disposed within housing 102, and a cover member 106 coupled to housing 102. In the exemplary embodiment, scent-releasing material 104 is a paradichlorobenzene (PDCB) material that sublimes in ambient air at room temperature to release a moth-repelling scent, and scent-releasing material 104 is provided in the form of two elongated tablets 108 or blocks sized for insertion into housing 102, as described in more detail below. In other embodiments, scent-releasing material 104 may be any suitable material having any suitable shape (e.g., scent-releasing material 104 may be provided in the form of four smaller tablets sized to be disposed within housing 102).

In the exemplary embodiment, housing 102 includes a body 110, a bottom cap 112, and a mounting mechanism (e.g., a hanger 114). Body 110 has a first end 116 (i.e., an open end) and a second end 118 (i.e., a closed end). Bottom cap 112 is coupled to body 110 at first end 116 such that bottom cap 112 covers first end 116 in a manner that seals tablets 108 within body 110 and in a manner that inhibits a user from detaching bottom cap 112 from body 110 (e.g., in one embodiment, bottom cap 112 is coupled to first end 116 via a suitable bonding operation such as, for example, an ultrasonic welding operation). In other embodiments, bottom cap 112 may be coupled to body 110 in any suitable manner that facilitates maintaining an air-tight seal between bottom cap 112 and body 110. In the exemplary embodiment, hanger 114 is integrally formed with, and extends outwardly from, body 110 at second end 118, enabling scent-releasing apparatus 100 to be hung in a suitable location at which the scent is to be released (e.g., in a closet). In other embodiments, housing 102 may have any suitable mounting mechanism (e.g., a bracket) rather than, or in combination with, hanger 114. In the exemplary embodiment, housing 102 is fabricated from a synthetic or semi-synthetic, organic-based material (e.g., a “plastic” material) using a molding process. It is understood, however, that housing 102 may be fabricated from any suitable material using any suitable manufacturing process.

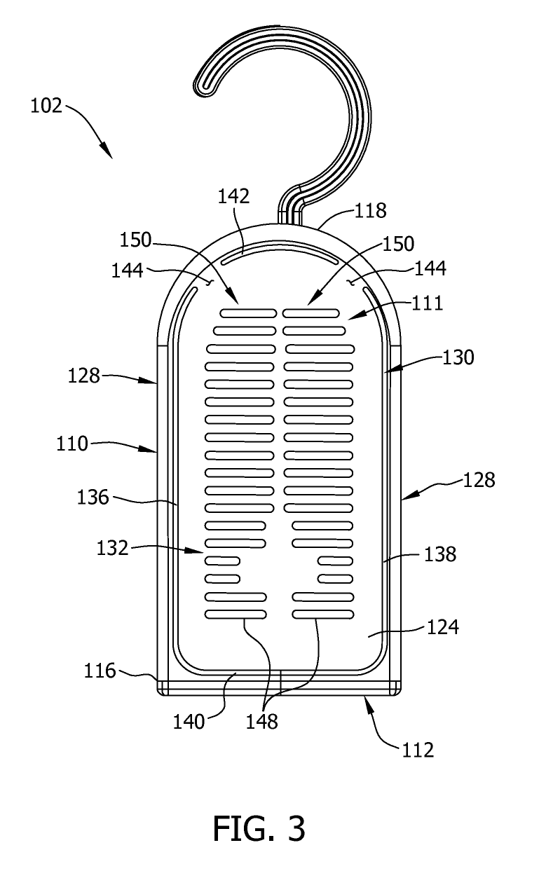

In the exemplary embodiment, cover member 106 is a product label (i.e., a label that displays branding and/or marketing indicia), and cover member 106 is removably attached to body 110 using an adhesive. Cover member 106 has at least one pull-tab 122 that enables a user to easily detach cover member 106 from body 110 when scent-releasing apparatus 100 is to be deployed, as described in more detail below. In one embodiment, cover member 106 has a low permeability (i.e., is substantially fluid impervious), and body 110 includes a contact area 111 (FIG. 3) with a polished surface for improved adherence of cover member 106 to housing 102. In another embodiment, cover member 106 is a multi-layered structure that is substantially flexible to permit cover member 106 to resiliently stretch in response to slight deformations of housing 102, while maintaining sufficient barrier properties that enable cover member 106 to remain substantially fluid impervious. In other embodiments, cover member 106 may not be a product label (i.e., cover member 106 may or may not include suitable indicia). Alternatively, cover member 106 may have any suitable properties (e.g., any suitable layering, flexibility, and/or permeability) that enable cover member 106 to function as described herein. As used herein, the term “scent-releasing material” refers to any material that is configured to release a scent or odor for a purpose such as deodorizing, insect repelling, or insect exterminating, and the term “substantially fluid impervious” refers to the ability of a material to inhibit gas and/or liquid from completely penetrating or passing through the material.

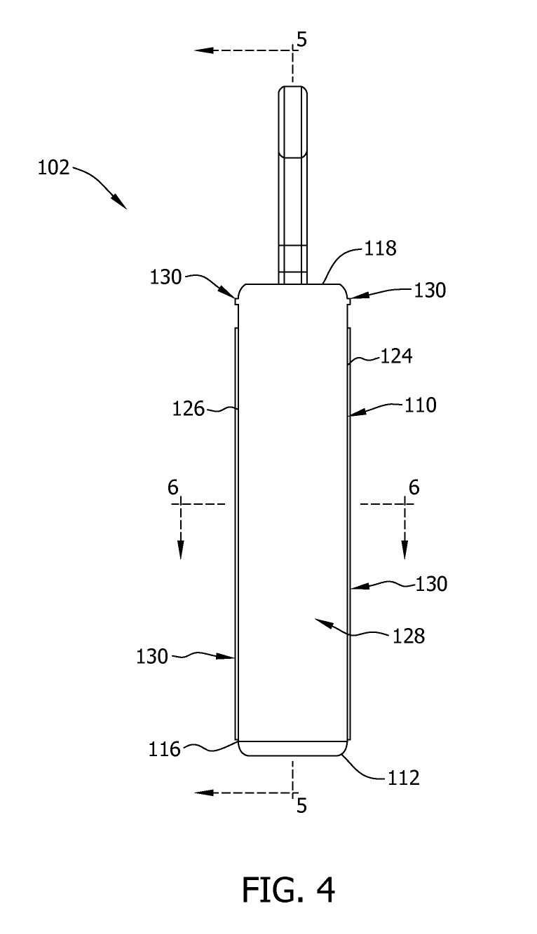

FIGS. 3 and 4 are front and side views, respectively, of housing 102 with bottom cap 112 coupled to body 110 and cover member 106 detached from body 110. In the exemplary embodiment, body 110 includes a front wall 124, a back wall 126, and a side wall 128. Front wall 124 and back wall 126 extend from first end 116 to second end 118, and side wall 128 extends between front wall 124 and back wall 126.

In the exemplary embodiment, a raised rib 130 extends outwardly from front wall 124 about a central region 132 of front wall 124, and a plurality of apertures 148 are arranged within central region 132. Raised rib 130 includes a first side portion 136, a second side portion 138 opposite first side portion 136, a lower portion 140 extending from first side portion 136 to second side portion 138, and an upper portion 142 disposed between first side portion 136 and second side portion 138. Upper portion 142 is arcuately shaped and is sized to not extend completely from first side portion 136 to second side portion 138, thereby defining at least one space 144 between upper portion 142 and first side portion 136 and/or second side portion 138. In other embodiments, raised rib 130 may have any suitable configuration (e.g., any suitable number of portions arranged in any suitable manner) that enables body 110 to function as described herein. In the exemplary embodiment, cover member 106 is sized for attachment to body 110 within central region 132 with space(s) 144 sized to receive pull-tabs 122 of cover member 106 (as shown in FIG. 1) such that raised rib 130 extends about at least a portion of an outer perimeter of cover member 106 to facilitate protecting cover member 106 during shipment of scent-releasing apparatus 100, as described in more detail below.

In the exemplary embodiment, apertures 148 are arranged in parallel rows 150 that are arranged side-by-side, and each row 150 contains apertures 148 that are substantially linear and are substantially parallel to one another. In other embodiments, apertures 148 may have any suitable shape (e.g., a curvilinear shape) and may be arranged in any suitable configuration on any suitable portion of body 110. In the exemplary embodiment, back wall 126 is substantially similar to front wall 124 (e.g., back wall 126 also includes raised rib 130 and apertures 148 arranged in a manner similar to that of front wall 124). Alternatively, back wall 126 may be different than front wall 124 (e.g., either front wall 124 or back wall 126 may not include raised rib 130 and/or apertures 148).

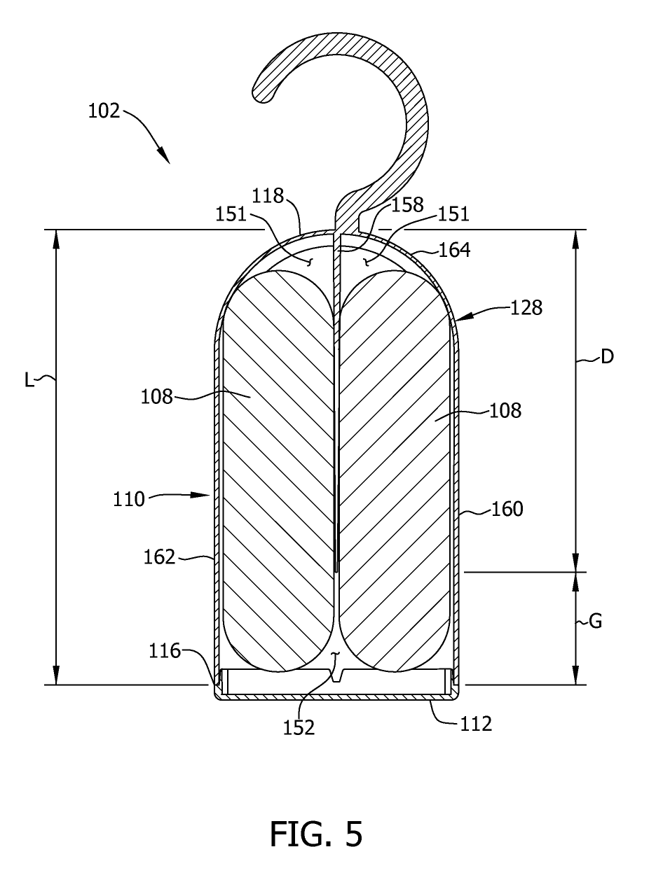

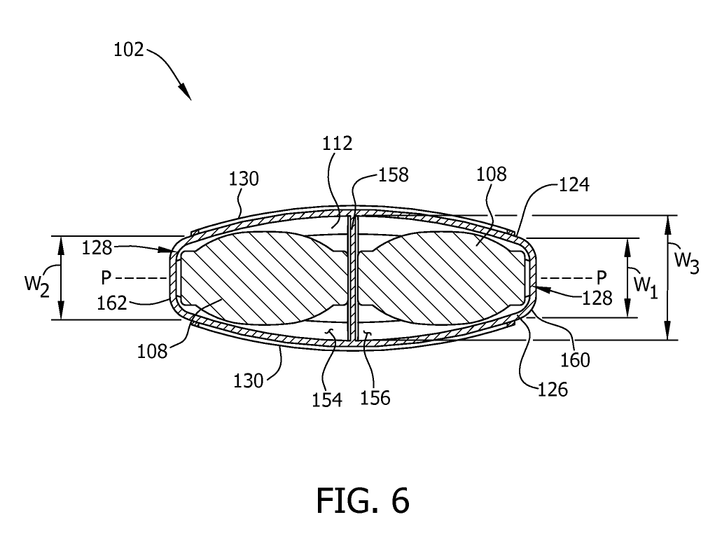

FIGS. 5 and 6 are cross-sectional views of housing 102 as shown in FIG. 4 and taken along lines 5-5 and 6-6, respectively. In the exemplary embodiment, housing 102 has an inner chamber 152 defined between front wall 124, back wall 126, side wall 128, and bottom cap 112. Inner chamber 152 is segmented into a first half 154 and a second half 156 by an inner reinforcing wall 158 that is integrally formed with side wall 128 at second end 118 and extends into inner chamber 152 toward first end 116. Additionally, inner reinforcing wall 158 is integrally formed with and extends from front wall 124 to back wall 126 between rows 150, aligning first half 154 with one row 150 of apertures 148 and second half 156 with the other row 150 of apertures 148. First half 154 is sized to receive one tablet 108, and second half 156 is sized to receive the other tablet 108 such that a headspace 151 is defined between tablets 108 and second end 118. Each tablet 108 is seated against side wall 128 and inner reinforcing wall 158 to prevent a movement of tablets 108 within inner chamber 152.

In the exemplary embodiment, body 110 has a length L from first end 116 to second end 118, and inner reinforcing wall 158 extends a distance D into inner chamber 152 from second end 118, wherein distance D is less than length L and defines a gap G between first end 116 and inner reinforcing wall 158. In other embodiments, inner reinforcing wall 158 may extend into inner chamber 152 any suitable distance (e.g., inner reinforcing wall 158 may extend from second end 118 to first end 116 with distance D and length L being substantially the same), and inner reinforcing wall 158 may have any suitable curvature oriented in any suitable manner within inner chamber 152. Alternatively, body 110 may not include inner reinforcing wall 158 (i.e., inner chamber 152 may not be segmented).

In the exemplary embodiment, side wall 128 has a first portion 160, a second portion 162 opposite first portion 160, and a third portion (i.e., a top portion 164) extending between first portion 160 and second portion 162. First portion 160 is substantially parallel to second portion 162, and first portion 160 and second portion 162 have a first width W₁ and a second width W₂, respectively. Similarly, inner reinforcing wall 158 is disposed between, and is oriented substantially parallel with, first portion 160 and second portion 162, and inner reinforcing wall 158 has third width W₃. First width W₁ and second width W₂ are substantially the same, and third width W₃ is greater than first width W₁ and second width W₂. Front wall 124 and back wall 126 extend from first portion 160 of side wall 128 to second portion 162 of side wall 128 around inner reinforcing wall 158, with inner reinforcing wall 158 integrally formed with front wall 124 and back wall 126. Because third width W₃ is greater than first width W₁ and second width W₂, front wall 124 and back wall 126 are contoured convexly outward relative to a plane P that extends through housing 102, wherein plane P is substantially perpendicular to first portion 160 and second portion 162 such that first half 154 and second half 156 of inner chamber 152 are generally frusto-conically shaped. In other embodiments, third width W₃ may not be greater than first width W₁ and/or second width W₂. Rather, third width W₃ may be less than or equal to first width W₁ and/or second width W₂ such that first half 154 and second half 156 of inner chamber 152 may have any suitable shape (i.e., such that front wall 124 and back wall 126 may have any suitable contours).

To deploy scent-releasing apparatus 100, a user grasps cover member 106 via pull-tab(s) 122 and detaches cover member 106 from housing 102 to permit airflow communication between the ambient air and inner chamber 152 through apertures 148. The ambient air sublimes tablets 108 to release a moth-repelling scent. The scent fills inner chamber 152 (e.g., fills headspace 151) and flows out of inner chamber 152 through apertures 148 and into the ambient air surrounding housing 102. With the moth-repelling scent being released from apertures 148, the scent-releasing apparatus 100 can be hung near clothing stored in a closet or other suitable space via hanger 114 in order to prevent moths from eating the clothing.

Because bottom cap 112 is bonded (e.g., ultrasonically welded) to housing 102 in an air-tight manner and because cover member 106 is removably attached to housing 102 over apertures 148, housing 102 is maintained substantially air-tight to prevent ambient air from entering inner chamber 152 until cover member 106 is removed, thereby preventing premature sublimation of the PDCB, increasing the shelf-life of the scent-releasing apparatus 100, and maintaining the production weight of the scent-releasing apparatus 100. Moreover, because cover member 106 is a multi-layered structure that is flexible and has adequate barrier properties and because central region 132 of body 110 has a contact area 111 with a polished surface for improved adherence of cover member 106 to housing 102, cover member 106 permits slight deformations of housing 102 at high temperatures without detaching from housing 102, thereby preventing premature sublimation of the PDCB. Additionally, raised rib 130 protects cover member 106 from damage during storage and/or shipping, which also prevents premature sublimation of the PDCB. Moreover, pressure may be created in housing 102 (e.g., in headspace 151) due to a vapor generated by the PDCB, and the curvilinear contour of housing 102 (e.g., convexly contoured front wall 124 and/or back wall 126) and inner reinforcing wall 158 provide sufficient support to prevent significant deformation of housing 102 as a result of the pressure.

The methods and systems described herein therefore facilitate providing a scent-releasing apparatus that can be easily deployed by simply removing a cover member attached to the outside of the housing, thereby preventing the user from having to handle the scent-releasing material. Also, the methods and systems described herein facilitate providing a scent-releasing apparatus with curvilinear outer walls and an inner reinforcing wall that enable the housing to better withstand pressure generated by the scent-releasing material. Moreover, the methods and systems described herein facilitate providing a raised rib on the housing about at least a portion of an outer perimeter of the cover member to protect the cover member from damage during storage and/or shipping, thereby increasing a shelf life of the scent-releasing apparatus while maintaining a production weight of the scent-releasing apparatus.

Exemplary embodiments of a scent-releasing apparatus are described above in detail. The scent-releasing apparatus described herein is not limited to the specific embodiments described herein, but rather, components of the apparatus may be utilized independently and separately from one another. For example, the apparatus described herein may have other applications not limited to practice with insect repellant, as described herein. Rather, the apparatus described herein can be implemented and utilized in connection with various other industries.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A scent-releasing apparatus comprising:
a housing comprising at least a front wall, a back wall, and a side wall connecting said front wall to said back wall, said housing defining an inner chamber, wherein at least one of said front wall and said back wall comprises a plurality of apertures;
at least one cover member removably attached to at least one of said front wall and said back wall for covering said plurality of apertures, said housing and said cover member configured to substantially seal the inner chamber from ambient air; and
a scent-releasing material positioned within the inner chamber, said scent-releasing material configured to release a scent through said apertures when said at least one cover member is removed from at least one of said front wall and said back wall.

2. A scent-releasing apparatus in accordance with claim 1, wherein said at least one cover member is flexible and multi-layered.

3. A scent-releasing apparatus in accordance with claim 1, wherein said at least one cover member is configured to create a substantially air-tight seal between the inner chamber and ambient air.

4. A scent-releasing apparatus in accordance with claim 1, wherein said housing further comprises a rib extending outwardly from said housing and about at least a portion of an outer perimeter of said at least one cover member, said rib configured to protect said at least one cover member during shipment of said apparatus.

5. A scent-releasing apparatus in accordance with claim 1, wherein said housing further comprises an inner reinforcing wall extending within the inner chamber between said front wall and said back wall, said inner reinforcing wall configured to prevent deformation of said housing.

6. A scent-releasing apparatus in accordance with claim 1, wherein said side wall further comprises a first side wall portion, a second side wall portion, and a top side wall portion, said housing further comprising a bottom cap ultrasonically welded to said front wall, said back wall, said first side wall portion, and said second side wall portion.

7. A scent-releasing apparatus comprising:
a scent-releasing material; and
a housing comprising a front wall, a back wall, and a side wall connecting said front wall to said back wall to define an inner chamber, said scent-releasing material being positioned within the inner chamber and substantially sealed from ambient air, wherein at least one of said front wall and said back wall is curvilinearly contoured with an inner reinforcing wall extending between said front wall and said back wall within the inner chamber.

8. A scent-releasing apparatus in accordance with claim 7, wherein said inner reinforcing wall bisects at least a portion of the inner chamber into a first half and a second half.

9. A scent-releasing apparatus in accordance with claim 7, wherein said housing further comprises a body having a length, said inner reinforcing wall extending into the inner chamber a distance that is less than the length of said body.

10. A scent-releasing apparatus in accordance with claim 7, wherein said housing further comprises a bottom cap ultrasonically welded to said front wall, said back wall, and said side wall.

11. A scent-releasing apparatus in accordance with claim 7, further comprising a plurality of apertures within at least one of said front wall and said back wall for providing airflow communication between the inner chamber and ambient air, and a removable cover member positionable over said plurality of apertures for sealing the inner chamber from ambient air, wherein said apparatus is configured to allow ambient air to enter the inner chamber when said cover member is removed and react with said scent-releasing material to release a scent into the ambient air.

12. A scent-releasing apparatus in accordance with claim 11, wherein said housing further comprises a rib extending outwardly from said housing and about at least a portion of an outer perimeter of said cover member, said rib configured to protect said cover member during shipment of said apparatus.

13. A scent-releasing apparatus in accordance with claim 11, wherein said housing further comprises a contact area comprising a polished surface to which said cover member is removably attached.

14. A scent-releasing apparatus in accordance with claim 7, wherein said housing further comprises a hanger.

15. A scent-releasing apparatus comprising:
a body comprising a plurality of apertures;
a bottom cap welded to said body to define an inner chamber between said body and said bottom cap;
a scent-releasing material disposed within the inner chamber; and
at least one substantially fluid impervious cover member removably attached to said body over said plurality of apertures to substantially seal the inner chamber from ambient airflow into the inner chamber through said plurality of apertures.

16. A scent-releasing apparatus in accordance with claim 15, wherein said bottom cap is ultrasonically welded to said body.

17. A scent-releasing apparatus in accordance with claim 15, wherein said body further comprises a rib extending outwardly from said body and about at least a portion of an outer perimeter of said at least one cover member, said rib configured to protect said at least one cover member during shipment of said apparatus.

18. A scent-releasing apparatus in accordance with claim 15, wherein said body further comprises a contact area comprising a polished surface to which said at least one cover member is removably attached.

19. A scent-releasing apparatus in accordance with claim 15, wherein said at least one cover member is flexible.

20. A scent-releasing apparatus in accordance with claim 15, wherein said apparatus further comprises a hanger.
